(19) United States
(12) Patent Application Publication
(10) Pub. No.: US 2024/0263319 A1
(43) Pub. Date: Aug. 8, 2024

(54) SYSTEM AND METHOD EMBODIMENTS FOR COMBINED ELECTROCHEMICAL CARBON DIOXIDE REDUCTION AND METHANOL OXIDATION

(71) Applicant: Battelle Memorial Institute, Richland, WA (US)

(72) Inventors: Michael R. Thorson, Richland, WA (US); Uriah Kilgore, Richland, WA (US); Udishnu Sanyal, Richland, WA (US); Karthikeyan Kallupalayam Ramasamy, Richland, WA (US)

(73) Assignee: Battelle Memorial Institute, Richland, WA (US)

(21) Appl. No.: 18/563,223

(22) PCT Filed: May 26, 2022
(86) PCT No.: PCT/US2022/031147

§ 371 Date: Nov. 21, 2023

Related U.S. Application Data
(60) Provisional application No. 63/193,958, filed on May 27, 2021.

(51) Int. Cl. C25B 3/07; C25B 3/23; C25B 3/26; C25B 9/19; C25B 11/046; C25B 11/065; C25B 15/08

(52) U.S. Cl. CPC C25B 3/07; C25B 3/23; C25B 3/26; C25B 9/19; C25B 11/046; C25B 11/065; C25B 15/083

(57) ABSTRACT

Disclosed herein are embodiments of a system and method that combine carbon dioxide reduction and methanol oxidation to provide an efficient path to formaldehyde production. The system and method embodiments are designed to rely on methanol oxidation to formaldehyde to provide the electrons needed to drive carbon dioxide reduction to carbon monoxide. The carbon monoxide obtained by carbon dioxide reduction is converted to the methanol used in the system/method via hydrogenation.

### CROSS REFERENCE TO RELATED APPLICATION

This application claims the benefit of and priority to the earlier filing date of U.S. Provisional Patent Application No. 63/193,958, filed on May 27, 2021, the entirety of which is incorporated herein by reference in its entirety.

### ACKNOWLEDGMENT OF GOVERNMENT SUPPORT

This invention was made with Government support under Contract DE-AC05-76RL01830 awarded by the U.S. Department of Energy. The Government has certain rights in the invention.

### FIELD

The present disclosure is directed to system and method embodiments for carrying out electrochemical reduction of carbon dioxide to carbon monoxide and electrochemical oxidation of methanol to formaldehyde in a concurrent process.

### BACKGROUND

Using methanol produced via carbon monoxide to produce formaldehyde is a technology that has been a key focus in many industries. Conventional methods to convert methanol to formaldehyde, however, often result in energetically inefficient over-oxidation of methanol to formaldehyde and other products beside formaldehyde. As such, purification is needed, which only adds to the cost and complexity of such conventional methods. Furthermore, conventional methods are not well-suited to efficiently use CO₂ as a feedstock. There exists a need in the art for more efficient methods for producing formaldehyde as well as methods that utilize CO₂ as a feedstock for product formation.

### SUMMARY

Disclosed herein are embodiments of a system, comprising: a split electrochemical cell comprising (i) an anode half-cell comprising an anode half-cell inlet, an anode half-cell outlet, and an anode suitable for oxidizing MeOH to formaldehyde and (ii) a cathode half-cell comprising a cathode half-cell inlet, a cathode half-cell outlet, and a cathode suitable for reducing CO₂ to CO; and a hydrogenation reactor comprising a catalyst component suitable for converting CO to MeOH, a reactor inlet, and a reactor outlet, wherein the hydrogenation reactor is fluidly coupled to the cathode half-cell and the anode half-cell such that fluid comprising CO that exits the cathode half-cell outlet is delivered to the reactor inlet and fluid comprising MeOH that exits from the reactor outlet is delivered to the anode half-cell inlet.

Also disclosed herein are embodiments of a method, comprising: reducing CO₂ to CO in a cathode half-cell of a split electrochemical cell using a cathode; hydrogenating the CO produced in the cathode half-cell to MeOH using a hydrogenation reactor; and oxidizing the MeOH from the hydrogenation reactor to formaldehyde in an anode half-cell of the split electrochemical cell using an anode that is electrochemically coupled with the cathode; wherein oxidizing the MeOH to the formaldehyde and reducing the CO₂ to the CO occur substantially simultaneously in the split electrochemical cell such that MeOH oxidation drives CO₂ reduction.

The foregoing and other objects and features of the present disclosure will become more apparent from the following detailed description, which proceeds with reference to the accompanying figures.

### BRIEF DESCRIPTION OF THE DRAWINGS

FIG. 1 is a schematic comparing an electrochemical method for making formaldehyde and ethylene glycol from CO₂, as described herein, with current production routes that rely on thermochemical techniques, such as wherein ethylene glycol is formed from (i) methanol (or “MeOH”) as derived from syngas or (ii) petroleum sources.

FIG. 2 provides a reaction diagram of the electrochemical reactions that can take place according to method embodiments described herein.

FIG. 3 provides a schematic illustrating the electrochemical coupling that takes place in disclosed method and system embodiments, whereby electrons from methanol oxidation to formaldehyde can be used to drive CO₂ reduction to CO, which in turn becomes the source of the methanol used in the oxidation step.

FIG. 4 provides a schematic illustration of an exemplary continuous flow split electrochemical cell for use in method and system embodiments described herein.

FIG. 5 provides a schematic illustration of an exemplary batch flow split electrochemical cell for use in method and system embodiments described herein.

FIG. 6 provides a schematic illustration of an exemplary continuous flow system wherein a split electrochemical cell is fluidly coupled to a hydrogenation reactor.

### DETAILED DESCRIPTION

### Overview of Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, molarities, voltages, capacities, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “introduce,” “flow,” or “provide” to describe the disclosed methods. These terms are high-level abstractions of the actual operations that are performed. The actual operations that correspond to these terms may vary depending on the particular implementation and are readily discernible by one of ordinary skill in the art.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Directions and other relative references (e.g., inner, outer, upper, lower, etc.) may be used to facilitate discussion of the drawings and principles herein, but are not intended to be limiting. For example, certain terms may be used such as “inside,” “outside,” “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

In order to facilitate review of the various embodiments of the disclosure, the following explanations of specific terms are provided:
Catalyst: A substance, usually present in small amounts relative to reactants, which increases the rate of a chemical reaction without itself being consumed or undergoing a chemical change. A catalyst also may enable a reaction to proceed under different conditions (e.g., at a lower temperature) than otherwise possible.
Cell: As used herein, a cell refers to an electrochemical device used for generating a voltage or driving a current that induces a chemical reaction. An exemplary cell according to the present disclosure is a split electrochemical cell that facilitates redox flow in a process wherein electrochemical oxidation of MeOH to formaldehyde is used to drive the reduction of CO₂ to CO.
Current density: A term referring to the amount of current per unit area. Current density is typically expressed in units of mA/cm².
Electrolyte: A substance containing free ions and/or radicals that behaves as an ionically conductive medium. In a redox flow reactor, some of the free ions and/or radicals are electrochemically active components. An electrolyte in contact with the anode, or anode half-cell, may be referred to as an anolyte, and an electrolyte in contact with the cathode, or cathode half-cell, may be referred to as a catholyte.
Faradic Efficiency: A measure of selectivity for an electrochemical process. In the context of CO₂ reduction to CO, faradic efficiency is defined as the fraction of total electrolysis current that is used towards reducing CO₂ to CO. In the context of MeOH oxidation to formaldehyde, faradic efficiency is defined as the fraction of total electrolysis current that is used towards oxidizing MeOH to formaldehyde.
Fluid: As used herein, fluid can mean liquid or gas.
Half-cell: An electrochemical cell includes two half-cells. Each half-cell comprises an electrode (or a portion of an electrode) and an electrolyte. In embodiments described herein, one half-cell (such as an anode half-cell) comprises an anode (or an anode portion of a unitary electrode) suitable for oxidizing MeOH to formaldehyde and the other half-cell (such as a cathode half-cell) comprises a cathode (or a cathode portion of a unitary electrode) suitable for reducing CO₂ to CO. In some embodiments, the two half-cells are electrochemically connected such that electrons can be exchanged.

### Introduction

While green routes to formaldehyde are possible in the art, such as the conversion of carbon dioxide (“CO₂”) and green hydrogen to methanol (“MeOH”) via an adaptation of existing methanol technology, and then to formaldehyde via existing processes, this route has a significant drawback in that two moles of water are generated for each mole of formaldehyde produced, thereby requiring substantial hydrogen utilization. This results in using 7-7.5 MW/t to generate a 37% yield of formaldehyde. Method and system embodiments of the present disclosure, however, require less energy input, including hydrogen utilization and thus more efficiently utilize renewable power to an extent that it is anticipated that less than half the power requirement can be achieved (<4 MW/t to yield 37% formaldehyde).

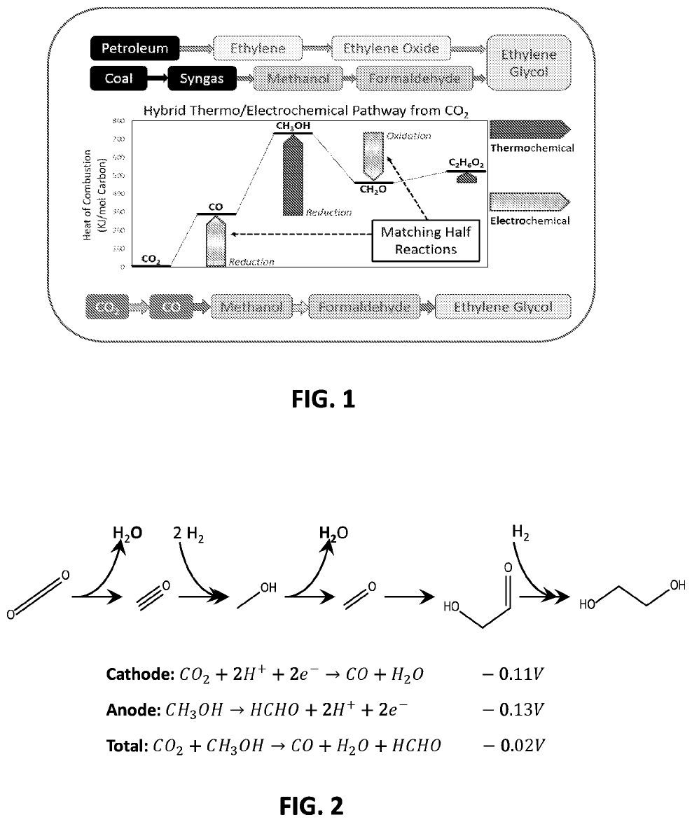

Additionally, compared to conventional routes to produce formaldehyde, the disclosed method and system embodiments also have the potential to reduce feedstock costs, thus providing an economically viable process (FIG. 1). The method and system embodiments disclosed herein can be used to convert CO₂ to formaldehyde using a combined electrochemical reduction/oxidation process. In embodiments of the disclosed method and system, CO₂ is reduced to carbon monoxide (“CO”), which is converted to MeOH. The MeOH produced in this step is then fed back into the process to drive the CO₂-to-CO reduction and simultaneously (or substantially simultaneously) is oxidized to formaldehyde. A reaction scheme illustrating the reactions that take place in some embodiments of the method is illustrated in FIG. 2. Solely by way of example, assuming faradaic efficiencies on the anode of the system (where MeOH is oxidized to formaldehyde) and the cathode (where CO₂ is reduced to CO) of 90%, and a hydrogenation yield of 95%, the overall feedstock component of the cost is anticipated to be $130/wet t, which is at least 17% lower than the conventional route. Furthermore, the disclosed method and system embodiments have a lower carbon footprint compared to conventional methods for making formaldehyde. For example, the feedstocks used in the disclosed method eliminates the carbon emissions associated with fossil feedstock production as well as those emitted from syngas generation associated with existing formaldehyde production. Additionally, the CO₂ emissions associated with the conversion of syngas to formaldehyde (which is 0.3 t CO₂/t formaldehyde) can be significantly reduced.

### Method Embodiments

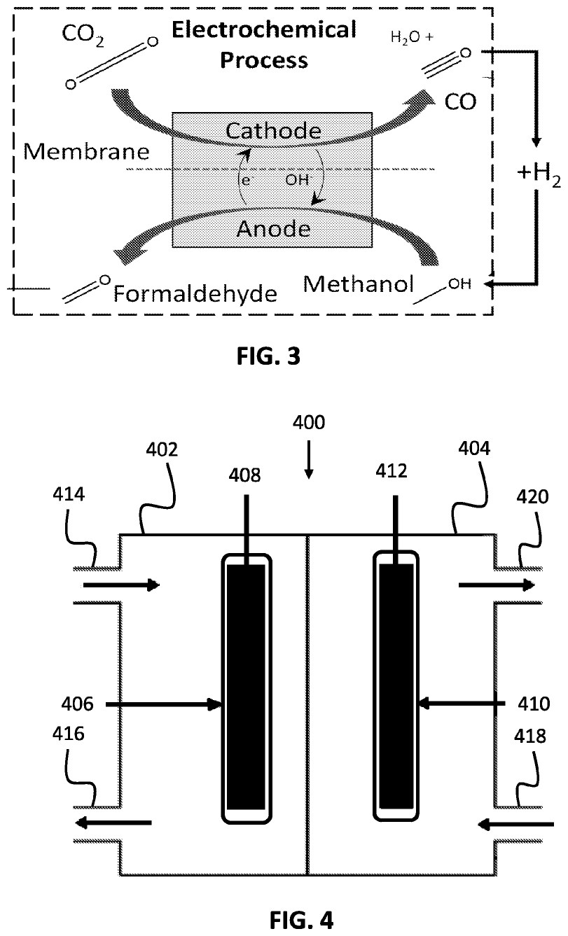

Disclosed herein are embodiments of a method for producing formaldehyde using a coupled reduction/oxidation electrochemical process. The method embodiments described herein utilize a combination of electrochemical reduction and oxidation to effectively convert CO₂ to formaldehyde. In particular embodiments, the method comprises introducing a CO₂ feedstock into a split electrochemical cell that comprises a cathode half-cell containing a cathode and an anode half-cell containing an anode, wherein the two half-cells are separated by a membrane or a separator component. The CO₂ feedstock is converted to CO at the cathode. The CO produced from reduction at the cathode can then be converted to methanol via a hydrogenation reaction. Methanol produced from this hydrogenation step is then fed back into the split electrochemical cell and is exposed to the anode whereby it is oxidized to formaldehyde. This oxidation in turn drives the reduction of the CO₂ to CO. An exemplary schematic is provided by FIG. 3. Additional features of the method embodiments are described herein.

### CO₂ Reduction to CO

Method embodiments disclosed herein comprise converting CO₂ to CO via reduction over a cathode. This reduction reaction occurs simultaneously, or at least substantially simultaneously with MeOH oxidation and within the same split electrochemical cell. The disclosed method embodiments efficiently produce CO from CO₂ and thus provide a feedstock for subsequent MeOH production.

A CO₂ feedstock is converted to CO by exposing the CO₂ to a cathode comprising a suitable reduction catalyst in the split electrochemical cell. The CO₂ feedstock can be introduced into the split cell in any suitable form, such as a gas or as a liquid. In particular embodiments, the CO₂ is introduced into the split electrochemical cell directly or indirectly from a CO₂ feedstock source. In some embodiments, the CO₂ can be obtained directly from a separate industrial facility (e.g., a syngas facility, a refinery, a petrochemical facility, or any suitable CO₂-capture facility) such that CO₂ is captured via the separate industrial facility and is funneled into the presently disclosed system. In other embodiments, the CO₂ feedstock used in the method can be captured at a CO₂ capture facility, stored, and then separately introduced into a system according to the present disclosure, which may be located at a geographically distinct location from that of the CO₂ capture facility.

The CO₂ feedstock can be directly fed into the electrochemical cell from the desired CO₂ source via a feed line that fluidly couples the CO₂ to the electrochemical cell. In other embodiments, the CO₂ can be fed from the CO₂ source to a vessel wherein it can be treated (e.g., purified and/or converted to a different form) prior to being introduced into the electrochemical cell. In some embodiments, the flow rate of the CO₂ into the split electrochemical cell can be controlled to provide a desired flow of CO₂ through the electrochemical cell so as to increase and/or decrease the amount of CO that is produced. In particular embodiments, the flow rate of CO₂ into the electrochemical cell and the amount of CO₂ used can be determined based on certain factors, such as input and output expectations. For example, in some embodiments, the flow rate of CO₂ and the amount of CO₂ used can be determined by considering the amount of formaldehyde to be produced. Solely by way of example, the amount of CO₂ used can be controlled so as to produce 0.0001% to 100% of the amount of formaldehyde used worldwide, which could comprise using an amount of CO₂ ranging from 0.0005 billion lb/yr to 5 billion lb/yr, such as 0.002 billion lb/yr to 1 billion lb/yr, or 0.01 billion lb/yr to 2 billion lb/yr, and a flow rate effective to facilitate up to 5 times the amount of CO₂ used, such as flow rates sufficient to use CO₂ amounts ranging from 0.002 billion lb/yr to 10 billion lb/yr, such as 0.01 billion lb/yr to 2 billion lb/yr, or 0.05 billion lb/yr to 0.5 billion lb/yr.

Upon introducing the CO₂ into the electrochemical cell, it can come into contact with (i) a catholyte solution, a solid electrolyte, or a combination thereof; and (ii) the cathode (which can be a gas diffusion cathode electrode or other suitable cathode). In some embodiments, the catholyte can be in the form of a solid ion-selective membrane, such as a sulfonated tetrafluoroethylene-copolymer membrane (e.g., Nafion®). In some embodiments, a catholyte solution is used, which can comprise a solvent (e.g., water or organic solvents) and a salt (e.g., KHCO₃, NaHCO₃). Suitable catholyte solutions for use in electrochemical CO₂ reduction to CO are known to those skilled in the art, particularly with the benefit of the present disclosure. The CO₂ can be introduced into the electrochemical cell as part of the catholyte solution or it can be introduced separate from the catholyte solution. In some particular embodiments, the CO₂ is dissolved in the catholyte solution or the solvent used for the catholyte solution. In some other embodiments, a gaseous stream of CO₂ can be introduced into the electrochemical cell, independent of any catholyte and/or catholyte solvent. The reduction process can be carried out under conditions suitable for controlling the reduction of the CO₂ to CO so as to, for example, avoid hydrogen evolution/production. In some embodiments, the reduction is carried out at atmospheric pressure or at a pressure that is above or below atmospheric pressure. The temperature used for the reduction can range from 20° C. to 200° C., such as 25° C. to 100° C., or 25° C. to 70° C. The voltage applied to the system to initiate CO₂ to CO reduction can range from 0 V to 3 V, such as 0.25 V to 1 V, or 0.25 V to 0.5 V.

### CO Hydrogenation to MeOH

Method embodiments disclosed herein comprise converting CO obtained from the reduction of CO₂ to MeOH to thereby provide the MeOH used to drive the CO₂ reduction. In particular embodiments of the method, the CO is hydrogenated to provide MeOH. The hydrogenation is carried out using a heterogeneous catalyst-based hydrogenation reaction. In some embodiments, CO produced in the split electrochemical cell can be passed to a separate hydrogenation reactor that houses a heterogeneous catalyst. In particular embodiments, the hydrogenation reactor can be a fixed bed reactor that can be operated batch-wise, or that can be operated continuously, such as with a fixed bed continuous-flow reactor. Once in the hydrogenation reactor, the CO exposed to the heterogeneous catalyst and H₂ gas under conditions that are known in the art to be sufficient to convert the CO to MeOH. In particular embodiments, this hydrogenation reaction can be carried out using a suitable heterogeneous catalyst system, which can be determined by those of ordinary skill in the art with the benefit of the present application. Suitable heterogeneous catalyst systems can include, but are not limited to zinc-based systems, copper-based systems, palladium-based systems, platinum-based systems, and nickel-based systems. In particular embodiments, the heterogeneous catalyst system can comprise ZnO—Cr₂O₃, Cu/ZnO, Cu/MgO, Cu/SiO₂, Pd/SiO₂, Cu/ZnO/Al₂O₃, or other catalyst systems known in the art. In some embodiments, the conditions suitable for hydrogenating the CO to MeOH can be selected based on the catalyst system that is used. In some embodiments using a ZnO—Cr₂O₃ catalyst system, the conditions can involve exposing the CO to the catalyst system in the presence of H₂ at a pressure ranging from 240 bar to 300 bar and a temperature ranging from 350° C. to 400° C. In yet additional embodiments using a Cu/ZnO catalyst system, the conditions can involve exposing the CO to the catalyst system in the presence of H₂ at a pressure ranging from 60 bar to 80 bar and at a temperature ranging from 250° C. to 280° C. MeOH produced from the hydrogenation step is then introduced back into the split electrochemical cell where it can be oxidized to formaldehyde as described herein. In some embodiments, the MeOH can first be treated prior to being introduced into the split electrochemical cell, but such treatment is not necessary.

### MeOH Oxidation to Formaldehyde

Method embodiments disclosed herein further comprise converting MeOH to formaldehyde. This oxidation reaction occurs simultaneously, or at least substantially simultaneously, with CO₂ reduction and within the same split electrochemical cell. The disclosed method embodiments produce formaldehyde from the MeOH with minimal to no over-oxidation of the MeOH to CO, CO₂, or formic acid. In some embodiments, no over-oxidation is observed. In some embodiments, the method yields less than 20%, such as less than 15%, less than 10%, less than 5%, or less than 2.5% formic acid, CO, and/or CO₂. Such yields can be for any individual over-oxidized product or for all such over-oxidized products. In particular embodiments, these yields correspond to the total amount of any over-oxidized products. In some embodiments, formic acid may be observed as a by-product; however, without being limited to a single theory, it currently is believed that such formic acid production may result from chemical reactions (e.g., a Cannizzaro reaction) of reaction products, rather than electrochemical conversions taking place at the electrode.

MeOH obtained in the method is converted to formaldehyde by exposing the MeOH to an anode comprising a suitable oxidizing catalyst in an anode half-cell of the split electrochemical cell. The MeOH can be introduced into the anode half-cell in any suitable form, such as a gas or as a liquid. In particular embodiments, the MeOH is introduced into the anode half-cell directly or indirectly from a separate hydrogenation reactor wherein the MeOH has been produced by hydrogenating CO produced from CO₂. The MeOH can be directly fed into the anode half-cell from the separate reactor via a feed line that fluidly couples the hydrogenation reactor to the split electrochemical cell. In other embodiments, the MeOH can be fed from the hydrogenation reactor to another vessel wherein it can be treated (e.g., purified, dried, and/or converted to a different form) prior to being introduced into the split electrochemical cell. In some embodiments, the flow rate of the MeOH into the split electrochemical cell can be controlled to provide a desired flow of MeOH through the electrochemical cell so as to increase and/or decrease (i) the amount of formaldehyde that is produced, (ii) the amount of CO₂ that is reduced to CO; or combinations thereof. In particular embodiments, the flow rate of MeOH into the electrochemical cell can be selected to match the number of moles from the CO₂ reduction step discussed above.

Upon introducing the MeOH into the electrochemical cell, it can come into contact with (i) an anolyte solution, a solid electrolyte, or a combination thereof; and (ii) an anode (which can be a gas diffusion anode electrode or other suitable anode). In some embodiments, the anolyte can be in the form of a solid ion-selective membrane, such as a sulfonated tetrafluoroethylene-copolymer membrane (e.g., Nafion®). In some embodiments, the anolyte solution can comprise water and a salt (e.g., NaOH, NaCl, KCl, NaBr, KBr, KOH, and the like) or an acid (e.g., HCl, HBr, or H₂SO₄). The MeOH can be introduced into the electrochemical cell as part of the anolyte solution or it can be introduced separate from the anolyte solution. The oxidation process can be carried out under conditions suitable for controlling the oxidation of the MeOH to formaldehyde such that over-oxidized products are avoided or maintained at minimal amounts. In some embodiments, the oxidation is carried out at atmospheric pressure or at a pressure above or below atmospheric pressure. The temperature used for the oxidation can range from 20° C. to 200° C., such as 25° C. to 100° C., or 25° C. to 70° C.

By integrating the MeOH oxidation to formaldehyde with the CO₂ reduction to CO, method embodiments described herein are able to use the MeOH oxidation to “pay” for the CO₂ reduction. For example, in some embodiments, combining the reduction and oxidation reactions in the split electrochemical cell drops the theoretical cell voltage to 0.02 V (see FIG. 2). As such, the method recovers the otherwise wasted oxidation potential from the oxidation of methanol to formaldehyde. In some embodiments of the disclosed method, the formaldehyde yield from methanol can range from 30% to 100%, or 50% to 100%, or 70% to 100%, such as 75% to 100%, or 80% to 100%, or 85% to 100%, or 90% to 100%, or 95% to 100%. In some embodiments, such yields can be obtained using a current density greater than 100 mA/cm². In some embodiments, the formaldehyde yield is greater than 90%. In particular embodiments, the method embodiments of the present disclosure can produce good faradic efficiencies, such as efficiencies ranging from 30% to 99% (or higher), such as 40% to 99%, 50% to 99%, 60% to 99%, or 70% to 99%, or 80% to 99%, or 90% to 99%.

### Additional Method Features

In some embodiments, the method can further comprise converting formaldehyde to ethylene glycol. In such embodiments, the formaldehyde obtained from the oxidation of the MeOH can be converted to glycolaldehyde, which can then be converted to ethylene glycol. In particular embodiments, these further chemical conversions can take place in a separate reactor and/or reactors from the electrochemical cell used in the system. For example, in some embodiments, formaldehyde produced by the electrochemical cell can be delivered to a fluidly coupled reactor that facilitates converting the formaldehyde to glycolaldehyde. The glycolaldehyde is then converted to ethylene glycol. As such, the disclosed method embodiments provide a lower-cost and lower-energy path to making ethylene glycol.

In particular embodiments, formaldehyde produced according to system and method embodiments of the present disclosure can be converted by ethylene glycol using a suitable hydroformylation process to first convert the formaldehyde to glycolaldehyde and then using a suitable hydrogenation process to convert the glycolaldehyde to ethylene glycol. In particular embodiments, the hydroformylation process can comprise exposing the formaldehyde to hydrogen gas and carbon monoxide in the presence of a catalyst. In some embodiments, the catalyst can be a rhodium catalyst, such as RhH(CO)(PPh₃)₃, Rh(CO)(PPh₃)₂, Rh(CO)₂PPh₃, [Rh(CO₂CH₃)₂PPh_{3]2}, Rh(CO₂CH₃)(CO)(PPh₃)₂, [Rh(C₈H₁₂)(PPh₃)₂]BPh₄, and the like. In some embodiments, solvents are used, such as amine solvents (e.g., DMF and the like). Exemplary conditions and reagents for this process are described, for example, in Journal of Organometallic Chemistry, 194 (1980), 113-123, the relevant portion of which is incorporated herein by reference. Such methods can further comprise hydrogenating the resulting glycolaldehyde to ethylene glycol using hydrogenation conditions known to those of skill in the art with the benefit of the present disclosure. In particular embodiments, the conditions can comprise exposing glycolaldehyde to hydrogen gas in the presence of a catalyst, such as a nickel catalyst. In some embodiments, hydroformylation and hydrogenation can be carried out in tandem within the same reactor. In other embodiments, hydroformylation and hydrogenation can be carried out sequentially in separate reactors.

In yet additional embodiments, formaldehyde produced according to system and method embodiments of the present disclosure can be converted by ethylene glycol via a carbonylation reaction wherein formaldehyde is carbonylated with carbon monoxide to yield glycolic acid. The glycolic acid can then be esterified and hydrogenated to provide ethylene glycol. In some embodiments, the carbonylation process can comprise exposing formaldehyde to carbon monoxide in the presence of a catalyst, such as a zeolite catalyst, an acidic ion exchange resin, and/or a heteropolyacid. Solvents can be used, such as sulfolane or other solvents known to those of skill in the art with the benefit of the present disclosure. In some embodiments, the zeolite catalyst can be selected from Zeolites HY, ZSM-5, mordenite, and the like. In other embodiments, the acidic ion exchange resin can be selected from Amberlyst-15, Amberlyst-70, and the like. In embodiments using a heteropolyacid, the heteropolyacid can be phosphotungstic acid or other heteropolyacids, such as those disclosed in Catalysis Communications 10 (2009) 678-681, the relevant portion of which is incorporated by reference. Exemplary conditions that can be used for carbonylation are described, for example, in Catal. Lett. (2011) 141:749-753, the relevant portion of which is incorporated herein by reference. The carbonylation reaction can be followed by esterifying the glycolic acid with methanol to provide methyl 2-hydroxyacetate and then hydrogenating the methyl 2-hydroxyacetate to provide ethylene glycol. Conditions to perform these transformation are known to those of skill in the art, particularly with the benefit of the present disclosure.

The disclosed method embodiments also provide a lower-cost and lower-energy path to making other products from formaldehyde, such as plastics (e.g., plastic bottles and polyoxymethylene plastics), fibers/resins (e.g., urea formaldehyde resins, melamine resins, phenol formaldehyde resins), polyfunctional alcohols (e.g., pentaerythritol, which is used to make paints and explosives), methylene diphenyl diisocyanate (which is an important component in polyurethane paints and foams), and hexamine (which is used in phenol-formaldehyde resins as well as in explosives).

In some embodiments, the method can be conducted as a batch-wise method or as a continuous flow method. In a batch-wise embodiment, the method can be conducted in a split electrochemical cell that houses a CO₂ solution in one region of the cell and an MeOH solution in a separate region. The MeOH solution of the batch-wise method can include MeOH obtained by from hydrogenation of CO produced from reducing the CO₂. In a continuous flow embodiment, MeOH and CO₂ feeds are continuously fed into the electrochemical cell in separated compartments. CO is produced from the CO₂ and is continuously delivered from the electrochemical cell to a hydrogenation reactor, which continuously feeds the MeOH produced from hydrogenating the CO back into the electrochemical cell. Formaldehyde is continuously expelled by the electrochemical cell upon oxidation of MeOH at the anode.

### System Embodiments

Disclosed herein are embodiments of system for producing formaldehyde from CO₂ using the coupled reduction/oxidation method described herein. In particular embodiments, the system comprises a split electrochemical cell that houses a cathode and an anode. The system can further comprise a hydrogenation reactor wherein CO produced from CO₂ is converted to methanol using a hydrogenation catalyst and hydrogen. In yet some further embodiments, the system can further comprise one or more separate vessels wherein further processing of reactants and/or reagents can take place. For example, some system embodiments can comprise separate vessels that can be used to purify, dry, and/or modify the phase of a particular reagent and/or reactant. In yet additional embodiments, the system can further comprise one or more further reactors that facilitate converting formaldehyde to ethylene glycol.

The split electrochemical cell of the system can comprise two half-cells, (i) an anode half-cell that houses an anode or an anode portion of a membrane electrode assembly (or “MEA”); and (ii) a cathode half-cell that houses a cathode or a cathode portion of an MEA. As such, the split electrochemical cell houses an anode component and a cathode component. The anode component of the split electrochemical cell can be a standalone anode component or it can be an anode portion of an MEA. The cathode component of the split electrochemical cell can be a standalone cathode component or it can be a cathode portion of an MEA. The split electrochemical cell further comprises a membrane or separator component. The membrane or separator component can be used to separate the two half-cells of the electrochemical cell and/or the anode and cathode; however, each is still capable of facilitating ion exchange between the anode and cathode components of the two half-cells. In some embodiments, the membrane can be an ion-exchange membrane. In some other embodiments, the separator component can be a porous separator. The anode half-cell housing the anode or anode portion of any MEA can further comprise an electrode tank that houses the anolyte used in converting the MeOH to formaldehyde. The cathode half-cell housing the cathode or cathode portion of any MEA can further comprise an electrode tank that houses the catholyte used in converting the CO₂ to CO.

In some embodiments, the anode is prepared for compatibility with the MeOH to formaldehyde conversion. In some embodiments, the anode can be made mixing a catalyst component with an ionomer and then deposited on a porous diffusion layer. In some embodiments, the anode comprises a noble metal component, such as a platinum-, gold-, silver-, palladium-, ruthenium-, rhodium-, iridium-, or osmium-containing catalyst (or any combinations or alloys thereof); a non-noble metal component, such as a molybdate-, nickel-, copper-, cobalt-, or vanadate-containing catalyst (or any oxides and/or alloys thereof); or a combination thereof. In an independent embodiment, the catalyst can be a silver-, gold-, or copper-containing catalyst that is promoted by a metal oxide (such as CeO₂, Co₃O₄, Mn₃O₄, or the like). In some embodiments, the catalyst can be in the form of a metal foil, wire, mesh, or other suitable form. The catalyst component can further comprise a support material, such as a carbon support material (e.g., activated carbons, carbon nanostructures, ordered mesoporous carbons, carbon spheres, graphene-based supports, and the like) or other conductive support material. In some embodiments, the anode comprises a platinum-based, ruthenium-based, rhodium-based, palladium-based, osmium-based, or iridium-based catalyst (or a combination thereof). In particular embodiments, the anode comprises Pt/Ru, Pt, Pt electrodeposited on carbon paper or carbon felt; Pt/Cu deposited on carbon paper; silver gauze, Pt/Ru on cloth; NiCu foil; NiCu wire; Pd/Ni gauze, RuO₂—MoO₃ nanoparticles, and the like. Suitable ionomer compositions are known to those of skill in the art, particularly with the benefit of the present disclosure. In some embodiments, the ionomer can comprise anion exchange ionomers (e.g., quaternized poly(terphenylene) ionomers, alkyltrimethylammonium (TMA), 1,2-dimethylimidazolium (DMIm), N-methylpiperidinium (Pip), poly(ether sulfone) ionomers, poly(phenylene oxide) ionomers, poly(arylene ether) ionomers, poly(phenylene) ionomers, poly(benzimidazolium) ionomers, poly(arylene alkylene) ionomers, poly(aryl piperidinium) ionomers, and the like); proton exchange ionomers (e.g., sulfonated polyimides, sulfonated polyetheretherketone ionomers (s-PEEK), perfluorinated sulfonic ionomers like Nafion®, and the like); or combinations thereof.

The cathode is prepared for compatibility with the CO₂ to CO conversion. In some embodiments, the cathode is suitable for use in low-temperature electrolysis. In particular embodiments, the cathode is a carbon-based electrode, such as a graphite-based electrode, a carbon nanotube-based electrode, a Teflon-based electrode, or the like. Other carbon materials that can be used for the cathode are known in the art and can include, without limitation graphene, carbon felt, carbon foam, and the like, as well as heteroatom-doped variations of such materials (e.g., nitrogen-doped graphene). The cathode can further comprise a catalyst component. The catalyst component can be selected from silver-based catalysts, gold-based catalysts, zinc-based catalysts, and palladium-based catalysts. In some embodiments, the catalyst component is selected from silver nanoparticles, palladium nanoparticles, zinc dendrites, or gold nanoparticles. In exemplary embodiments, the cathode can comprise silver nanoparticles dispersed on multi-walled carbon nanotubes, gold nanoparticles dispersed on polymer-wrapped carbon nanotubes (e.g., poly(2,2′-(2,6-pyridine)-5,5′-bibenzimidazole), or “PyPBI”), or silver-coated Teflon. The catalyst component loading can be selected so as to provide efficient conversion of CO₂ to CO. In some embodiments, the catalyst component loading can range from 1 mg/cm² to 250 mg/cm². In yet some additional embodiments, the cathode is a gas diffusion electrode. In particular embodiments, the cathode can be a gas diffusion electrode as described in ACS Energy Lett. 2019, 4, 1, 317-324, wherein the gas diffusion electrode embodiments described therein are incorporated herein by reference.

The split electrochemical cell may be assembled in ambient atmosphere in a housing that is closed and operated with or without flowing an inert gas through the housing. In some embodiments, the housing may be sealed such that additional oxygen from the ambient atmosphere is excluded or substantially excluded and/or the components of the electrochemical cell can be oxyphobic. During operation, the catholyte and anolyte can be continuously circulated via pumps through the appropriate half-cell. The split electrochemical cell can further comprise an anode half-cell inlet and an anode half-cell outlet that facilitate fluid delivery into and out of the anode half-cell. The split electrochemical cell can also further comprise a cathode half-cell inlet and a cathode half-cell outlet that facilitate delivery into and out of the cathode half-cell. Other connections, ports, and/or inlet/outlets may be included with the split electrochemical cell so as to facilitate operation, along with other electrochemical components, such as a counter electrode, a reference electrode, or any combination thereof.

An exemplary continuous flow split-cell electrochemical cell is illustrated in FIG. 4. As illustrated in FIG. 4, cell 400 comprises anode half-cell 402 and cathode half-cell 404. Anode half-cell 402 comprises anode compartment 406 that houses anode 408. Cathode half-cell 404 comprises cathode compartment 410, which houses cathode 412. Anode half-cell 402 further comprises anode half-cell inlet 414 and anode half-cell outlet 416. Cathode half-cell 404 further comprises cathode half-cell inlet 418 and cathode half-cell outlet 420.

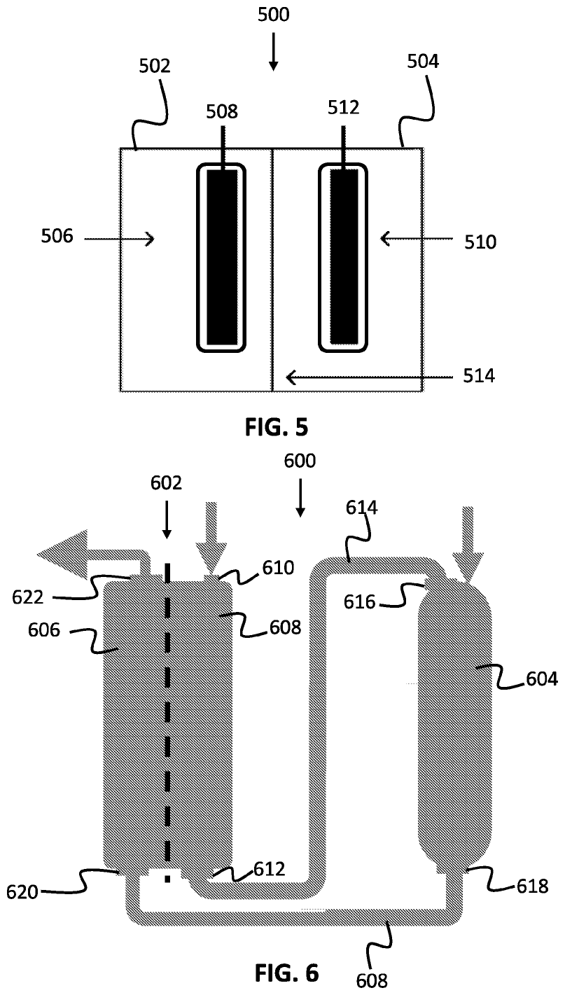

An exemplary batch electrochemical cell is illustrated in FIG. 5. As illustrated in FIG. 5, cell 500 comprises anode half-cell 502 and cathode half-cell 504. Anode half-cell 502 comprises anode compartment 506 that houses anode 508. Cathode half-cell 504 comprises cathode compartment 510, which houses cathode 512. Cell 500 further comprises a membrane component, membrane 514, which separates anode half-cell 502 and cathode half-cell 504.

In particular embodiments, the system comprises at least the split electrochemical cell and the hydrogenation reactor. In such embodiments, the hydrogenation reactor is fluidly coupled to the split electrochemical cell such that CO produced from the cathode-containing portion of the electrochemical cell (the second half cell) can be delivered to the hydrogenation reactor where it is converted to MeOH. In particular embodiments, the hydrogenation reactor comprises a reactor inlet and a reactor outlet and is fluidly coupled to the cathode half-cell and the anode half-cell such that fluid exiting the cathode half-cell outlet is delivered to the reactor inlet and fluid from the reactor outlet is delivered to the anode half-cell inlet. The hydrogenation reactor and the split electrochemical cell are further fluidly coupled such that the MeOH from the hydrogenation reactor can be delivered to the anode-containing half of the electrochemical cell (the first half cell). In some embodiments, fluid connectivity between the split electrochemical cell and the hydrogenation reactor can be maintained through hoses, connecting lines, or other physical means for transferring fluids between the two components. In particular embodiments, the hydrogenation reactor is a fixed bed continuous-flow reactor. The hydrogenation reactor can comprise components capable of hydrogenating CO to methanol according to method embodiments described herein. In some embodiments, the hydrogenation reactor houses a support material and/or a hydrogenation catalyst as described herein and is connected to a hydrogen gas source.

FIG. 6 provides a schematic illustration of an exemplary system comprising the split-electrochemical cell and a fluidly coupled hydrogenation reactor. System 600, as illustrated in FIG. 6, comprises split electrochemical cell 602, which is fluidly coupled to hydrogenation reactor 604. Split electrochemical cell 602 comprises anode half-cell 606, which houses the anode portion of the device (not illustrated), and cathode half-cell 608, which houses the cathode portion of the device (not illustrated). Cathode half-cell 608 comprises cathode half-cell inlet 610, which allows for CO₂ delivery into the system, along with half-cell outlet 612, which facilitates fluid delivery of CO to hydrogenation reactor 604 via connecting line 614. The CO is allowed to enter hydrogenation reactor 604 via reactor inlet 616, where it is converted to MeOH. MeOH leaves hydrogenation reactor 604 via connecting line 618 and is passed to anode half-cell inlet 620. In anode half-cell 606, the MeOH is oxidized to formaldehyde, which can then exit the system via anode half-cell outlet 622.

In some embodiments, the system can further comprise reactors used to convert formaldehyde generated according to the disclosed method embodiments to ethylene glycol. The reactors used for these additional transformations can be fluidly coupled to the anode half-cell outlet of the split electrochemical cell such that formaldehyde can be expelled from the anode half-cell into any such reactors. In some such embodiments, the system can further comprise a reactor suitable for hydroformylation of formaldehyde to glycolaldehyde; a reactor suitable for hydrogenation of glycolaldehyde to ethylene glycol; a reactor suitable for carbonylation of formaldehyde to glycolic acid; a reactor suitable for esterifying glycolic acid to methyl 2-hydroxyacetate; a reactor suitable for hydrogenating methyl 2-hydroxyacetate to ethylene glycol; and any combination of such reactors. In some embodiments, transformations of formaldehyde described herein can be conducted in the same reactor and/or steps of the transformations can be carried out in separate reactors. Exemplary reactors suitable for any of the formaldehyde to ethylene glycol transformations are recognized by those skilled in the art, particularly with the benefit of the present disclosure.

### Overview of Several Examples

Disclosed herein are embodiments of a system, comprising: a split electrochemical cell comprising (i) an anode half-cell comprising an anode half-cell inlet, an anode half-cell outlet, and an anode suitable for oxidizing MeOH to formaldehyde and (ii) a cathode half-cell comprising a cathode half-cell inlet, a cathode half-cell outlet, and a cathode suitable for reducing CO₂ to CO; and a hydrogenation reactor comprising a catalyst component suitable for converting CO to MeOH, a reactor inlet, and a reactor outlet, wherein the hydrogenation reactor is fluidly coupled to the cathode half-cell and the anode half-cell such that fluid comprising CO that exits the cathode half-cell outlet is delivered to the reactor inlet and fluid comprising MeOH that exits from the reactor outlet is delivered to the anode half-cell inlet.

In some embodiments, the split electrochemical cell houses a membrane that is positioned between the anode half-cell and the cathode half-cell.

In any or all of the above embodiments, the anode and the cathode are provided as separate electrodes.

In any or all of the above embodiments, the anode and the cathode are provided as a membrane electrode assembly.

In any or all of the above embodiments, the anode half-cell comprises an anolyte and the cathode half-cell comprises a catholyte.

In any or all of the above embodiments, the system further comprises a further reactor fluidly coupled to the split electrochemical cell.

In any or all of the above embodiments, further reactor is fluidly coupled to the anode half-cell outlet such that fluid expelled from the anode half-cell outlet is delivered to an inlet of the further reactor.

In any or all of the above embodiments, the further reactor comprises:
(i) a catalyst that promotes hydroformylation of formaldehyde to glycolaldehyde;
(ii) a catalyst that promotes hydroformylation of formaldehyde to glycolaldehyde; and a catalyst and a support material that promotes hydrogenation of glycolaldehyde to ethylene glycol;
(iii) a catalyst that promotes carbonylation of formaldehyde to glycolic acid;
(iv) reagents that promote esterification of glycolic acid to methyl 2-hydroxyacetate;
(v) a catalyst and a support material that promotes hydrogenation of methyl 2-hydroxyacetate to ethylene glycol; or
(vi) any combination of (i)-(v).

Also disclosed herein are embodiments of a method, comprising: reducing CO₂ to CO in a cathode half-cell of a split electrochemical cell using a cathode; hydrogenating the CO produced in the cathode half-cell to MeOH using a hydrogenation reactor; and oxidizing the MeOH from the hydrogenation reactor to formaldehyde in an anode half-cell of the split electrochemical cell using an anode that is electrochemically coupled with the cathode; wherein oxidizing the MeOH to the formaldehyde and reducing the CO₂ to the CO occur substantially simultaneously in the split electrochemical cell such that MeOH oxidation drives CO₂ reduction.

In any or all of the above embodiments, the cathode comprises a catalyst component and a support component.

In any or all of the above embodiments, the catalyst component is selected from silver, gold, palladium and the support component is a carbon-based material.

In any or all of the above embodiments, the anode comprises a platinum-, gold-, ruthenium-, rhodium-, iridium-, or osmium-containing catalyst, or any combination of such catalysts.

In any or all of the above embodiments, the anode comprises a Pt/Ru foil.

In any or all of the above embodiments, the MeOH oxidation drives the CO₂ reduction by providing the electrons needed to electrochemically reduce the CO₂ to CO.

In any or all of the above embodiments, the method is performed continuously.

In any or all of the above embodiments, the method is performed batch-wise.

In any or all of the above embodiments, the CO₂ is provided by a CO₂ source.

In any or all of the above embodiments, the CO₂ source is a syngas facility.

In any or all of the above embodiments, the method further comprises transforming the formaldehyde to ethylene glycol.

In any or all of the above embodiments, the formaldehyde is transformed to ethylene glycol using (i) hydroformylation and hydrogenation; or (ii) carbonylation, esterification, and hydrogenation.

### EXAMPLES

### Example 1

In this example, methanol oxidation experiments were conducted in a 3-electrode configuration using an Ametek VersaSTAT 4 potentiostat and an H-Cell with a total volume of 50 mL. Methanol (approximately 2.5 M) and NaOH (0.5 M), in water solutions, were placed in the anode compartment while NaOH (0.5 M) in water solution was placed in the cathode compartment. A platinum wire was used as the counter electrode. The reference electrode was an aqueous Ag/AgCl wire in a glass compartment with a vycor plug. A 2.5 cm*2.5 cm Pt/Ru foil was used as the anode

The cell was subject to chronoamperometry at various potentials and time periods. The products were analyzed by liquid chromatograph using a small sample was removed from the anode compartment.

The potential was set to 0.5V (v. open circuit) and the current was collected for 1300 seconds. The faradaic efficiency of methanol oxidation in this example was found to be 31%.

After stopping the cell for sampling the anode compartment, chronoamperometry was continued at a potential of −0.1V (v. open circuit) for an additional 1083 seconds. The products were sampled and the faradaic efficiency with respect to formaldehyde was found to be 47%.

After stopping the cell for sampling the anode compartment, chronoamperometry was continued at a potential of −0.2V (v. open circuit) for an additional 3000 seconds. The products were sampled and the faradaic efficiency with respect to formaldehyde was found to be 66%.

### Example 2

In this example, methanol oxidation experiments were conducted in a 3-electrode configuration using an Ametek VersaSTAT 4 potentiostat and an H-Cell with a total volume of 50 mL. Methanol (approximately 2.5 M) and H₂SO₄ (0.5 M), in water solutions, were placed in the anode compartment while H₂SO₄ (0.5 M) in water solution was placed in the cathode compartment. A platinum wire was used as the counter electrode. The reference electrode was an aqueous Ag/AgCl wire in a glass compartment with a vycor plug. A platinum foil was used as the anode at a potential of 0.6V (v. Ag/AgCl reference). Current was collected for 2700 seconds. The faradaic efficiency toward formaldehyde was found to be 38% in this example.

### Example 3

In this example, an exemplary system comprising a split electrochemical fuel cell and a hydrogenation reactor suitable for continuous flow operation as described herein is evaluated. The split electrochemical fuel cell comprises an anode half-cell containing an anode and a cathode half-cell containing a cathode. The anode and the cathode can be as described herein. An anolyte as described herein is added to the anode half-cell and a catholyte as described herein is added to the cathode half-cell. A gaseous stream of CO₂ is introduced into the cathode half-cell at a suitable flow rate via an inlet and is allowed to contact the cathode. CO produced from the electrochemical reduction of the CO₂ is then directed from the cathode half-cell to a hydrogenation reactor via a connecting line that fluidly connects a cathode half-cell outlet to an inlet of the hydrogenation reactor. The CO is allowed to react with the hydrogenation catalyst present in the hydrogenation reactor and is converted to MeOH. The MeOH is then fed back into the split electrochemical fuel cell via a connecting line that fluidly connects an outlet of the hydrogenation reactor to the anode half-cell inlet. The MeOH undergoes electrochemical oxidation within the anode half-cell at the anode and the formaldehyde obtained therefrom is directed out of the anode half-cell via the anode half-cell outlet. The split electrochemical fuel cell is operated at a suitable potential, such as a potential of 0.5 V. In some examples, the conditions and reagents described in any of Examples 1 or 2 can be used in this example.

In view of the many possible embodiments to which the principles of the present disclosure may be applied, it should be recognized that the illustrated embodiments are only preferred examples of the disclosure and should not be taken as limiting the scope of the disclosure. Rather, the scope is defined by the following claims. We therefore claim as our invention all that comes within the scope and spirit of these claims.

1. A system, comprising:
a split electrochemical cell comprising (i) an anode half-cell comprising an anode half-cell inlet, an anode half-cell outlet, and an anode suitable for oxidizing MeOH to formaldehyde and (ii) a cathode half-cell comprising a cathode half-cell inlet, a cathode half-cell outlet, and a cathode suitable for reducing CO₂ to CO; and
a hydrogenation reactor comprising a catalyst component suitable for converting CO to MeOH, a reactor inlet, and a reactor outlet, wherein the hydrogenation reactor is fluidly coupled to the cathode half-cell and the anode half-cell such that fluid comprising CO that exits the cathode half-cell outlet is delivered to the reactor inlet and fluid comprising MeOH that exits from the reactor outlet is delivered to the anode half-cell inlet.

2. The system of claim 1, wherein the split electrochemical cell houses a membrane that is positioned between the anode half-cell and the cathode half-cell.

3. The system of claim 1, wherein the anode and the cathode are provided as separate electrodes.

4. The system of claim 1, wherein the anode and the cathode are provided as a membrane electrode assembly.

5. The system of claim 1, wherein the anode half-cell comprises an anolyte and the cathode half-cell comprises a catholyte.

6. The system of claim 1, further comprising a further reactor fluidly coupled to the split electrochemical cell.

7. The system of claim 6, wherein the further reactor is fluidly coupled to the anode half-cell outlet such that fluid expelled from the anode half-cell outlet is delivered to an inlet of the further reactor.

8. The system of claim 6, wherein the further reactor comprises:
(i) a catalyst that promotes hydroformylation of formaldehyde to glycolaldehyde;
(ii) a catalyst that promotes hydroformylation of formaldehyde to glycolaldehyde; and a catalyst and a support material that promotes hydrogenation of glycolaldehyde to ethylene glycol;
(iii) a catalyst that promotes carbonylation of formaldehyde to glycolic acid;
(iv) reagents that promote esterification of glycolic acid to methyl 2-hydroxyacetate;
(v) a catalyst and a support material that promotes hydrogenation of methyl 2-hydroxyacetate to ethylene glycol; or
(vi) any combination of two or more of (i)-(v).

9. A method, comprising:
reducing CO₂ to CO in a cathode half-cell of a split electrochemical cell using a cathode;
hydrogenating the CO produced in the cathode half-cell to MeOH using a hydrogenation reactor; and
oxidizing the MeOH from the hydrogenation reactor to formaldehyde in an anode half-cell of the split electrochemical cell using an anode that is electrochemically coupled with the cathode;
wherein oxidizing the MeOH to the formaldehyde and reducing the CO₂ to the CO occur substantially simultaneously in the split electrochemical cell such that MeOH oxidation drives CO₂ reduction.

10. The method of claim 9, wherein the cathode comprises a catalyst component and a support component.

11. The method of claim 10, wherein the catalyst component is selected from silver, gold, palladium and the support component is a carbon-based material.

12. The method of claim 9, wherein the anode comprises a platinum-, gold-, ruthenium-, rhodium-, iridium-, or osmium-containing catalyst, or any combination of such catalysts.

13. The method of claim 12, wherein the anode comprises a Pt/Ru foil.

14. The method of claim 9, wherein the MeOH oxidation drives the CO₂ reduction by providing the electrons needed to electrochemically reduce the CO₂ to CO.

15. The method of claim 9, wherein the method is performed continuously.

16. The method of claim 9, wherein the method is performed batch-wise.

17. The method of claim 9, wherein the CO₂ is provided by a CO₂ source.

18. The method of claim 17, wherein the CO₂ source is a syngas facility.

19. The method of claim 9, wherein the method further comprises transforming the formaldehyde to ethylene glycol.

20. The method of claim 19, wherein the formaldehyde is transformed to ethylene glycol using (i) hydroformylation and hydrogenation; or (ii) carbonylation, esterification, and hydrogenation.
